(19) United States
(12) Patent Application Publication
(10) Pub. No.: US 2016/0030716 A1
(43) Pub. Date: Feb. 4, 2016

(54) NEEDLE IN CATHETER CANNULATION SYSTEMS AND METHODS

(71) Applicant: THE UNIVERSITY OF UTAH RESEARCH FOUNDATION, Salt Lake City, UT (US)

(72) Inventors: Michael Mallin, Salt Lake City, UT (US); Michael Stone, Cambridge, MA (US)

(73) Assignee: THE UNIVERSITY OF UTAH RESEARCH FOUNDATION, Salt Lake City, UT (US)

(21) Appl. No.: 14/776,437

(22) PCT Filed: Mar. 5, 2014
(86) PCT No.: PCT/US14/20832

§ 371 Date: Sep. 14, 2015

Related U.S. Application Data
(60) Provisional application No. 61/781,788, filed on Mar. 14, 2013.

(51) Int. Cl. A61M 25/06; A61M 25/09

(52) U.S. Cl. CPC A61M 25/0606; A61M 25/09041; A61M 25/0612

(57) ABSTRACT

A cannulation system which may comprise a needle disposed within a catheter is disclosed. The needle may be coupled to a needle wire to facilitate displacement of the needle within the catheter. In some embodiments, a guidewire may be disposed within the needle and/or the catheter. This system and associated methods may facilitate needle placement and vascular cannulation.

### RELATED APPLICATIONS

This application claims priority to U.S. Provisional Application No. 61/781,788, titled “Needle in Catheter Cannulation Systems and Methods,” filed on Mar. 14, 2013, which is hereby incorporated by reference in its entirety.

### TECHNICAL FIELD

The present disclosure relates generally to medical devices, including catheters and cannulation kits or systems. Specifically, the present disclosure relates to systems and methods of cannulation through use of a needle disposed within a catheter.

### BRIEF DESCRIPTION OF THE DRAWINGS

The embodiments disclosed herein will become more fully apparent from the following description and appended claims, taken in conjunction with the accompanying drawing. This drawing depicts only a typical embodiment, which will be described with additional specificity and detail in connection with the figures described below.

FIG. 1 is a perspective view of a needle in a catheter assembly partially disposed within a vascular lumen of a patient.

FIG. 2 is an enlarged section view taken around section 2-2 of a needle in a catheter assembly of FIG. 1.

FIG. 3 is an enlarged section view taken around section 3-3 of a needle in a catheter assembly of FIG. 1.

### BACKGROUND

Vascular access procedures such as central venous cannulation may be used in connection with various therapies, such as: administration of crystalloid; monitoring of hemodynamic parameters; introduction of devices such as cardiac pacemakers, stents, filters, or other medical appliances; angioplasty; drug delivery, including vasoactive drug delivery; and so forth. Misplacement or other errors during cannulation may be associated with a variety of serious complications.

The kit commonly used for central venous cannulation has remained virtually unchanged over time. Traditional cannulation procedures utilize the Seldinger technique—the standard technique for “blind” cannulation based on anatomic landmarks. The Seldinger technique typically first includes the step of placement of a large-bore needle into the target vein of a patient. The practitioner conducting the procedure watches to observe a “flash” of blood in a syringe attached to the needle, indicating that the needle is located within the target vein. Following this, the syringe is removed by the practitioner, and a wire is introduced through the needle into the vein. The needle is then removed and an introducer and catheter are placed over the wire and into the vein.

This procedure depends on the presence of a “flash” of blood in the syringe to confirm proper needle placement. Ultrasound guidance has been used during needle placement procedures to attempt to remove the need to rely on the presence of the flash of blood or other anatomical landmarks. In many cases, however, visibility may be deficient, and later steps in the procedure still introduce risk to patients and practitioners. Specifically, subsequent to the needle placement step, the technique requires various steps that introduce opportunities to dislodge the needle from the vessel and expose the practitioner to potential needlesticks. For example, removal of the syringe may dislodge the needle tip from the vein, requiring the procedure to be restarted.

### DETAILED DESCRIPTION

A cannulation system is described herein which may comprise a needle disposed within a catheter. The needle may be coupled to a needle wire to allow displacement of the needle within the catheter. In some embodiments, a guidewire may be disposed within the needle and/or the catheter. This system and associated methods are adapted to facilitate needle placement into the vascular lumen of a patient and to assist in accurate, safe and efficient vascular cannulation by a medical practitioner.

In some procedures disclosed herein, ultrasound guidance may be used during needle placement to potentially obviate the need to rely on the presence of a “flash” of blood or other anatomical landmarks during needle placement. Additionally, the present disclosure also relates to systems and methods which may reduce the number of steps during cannulation and/or reduce the risk of error during a procedure.

As further detailed below, an assembly comprising a needle disposed within a catheter may be utilized during a cannulation procedure or other vascular access procedure. The systems and methods described herein may be associated with superior dexterity as well as less risk of error, for example, by eliminating the need for certain awkward or error-prone steps. Thus, the present disclosure may lessen the risk of certain complications such as arterial punctures, needle dislodgement, wire dislodgement, inability to pass the catheter, infection, needlesticks, and general procedure failure.

The components of the embodiments, as generally described and illustrated in the figures herein, could be arranged and designed in a variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless so indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device. As used herein, the proximal end of a medical device is defined as the end of the device configured to be disposed nearest the practitioner when the device is being manipulated or utilized. The distal end is the opposite end of the device, or the end furthest from the practitioner while the device is in use.

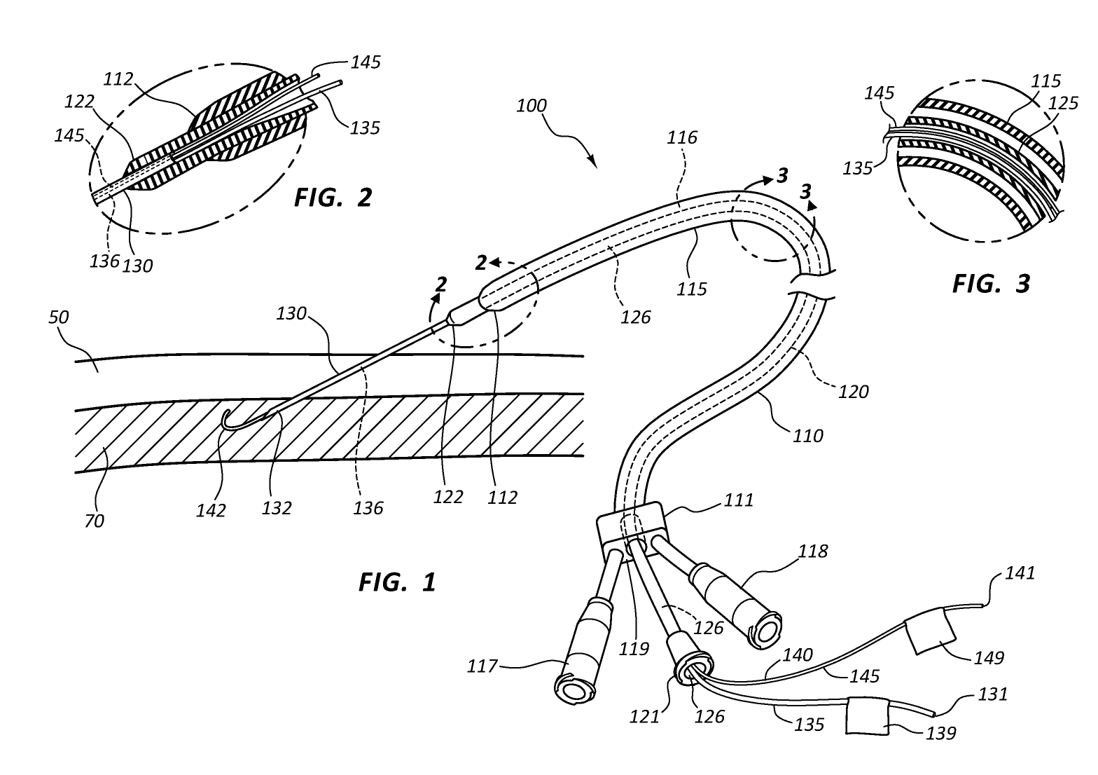

FIG. 1 is a perspective view of a needle in catheter assembly 100 partially disposed within a vascular lumen 50. FIGS. 2 and 3 are enlarged section view, taken around sections 2-2 and 3-3, respectively, of FIG. 1. As shown in FIGS. 1-3, in the illustrated embodiment, the assembly 100 comprises a catheter 110 extending from a proximal end 111 to a distal end 112 with a catheter body 115 disposed there between. The catheter 110 may comprise a catheter lumen 116 disposed within the catheter 110. The catheter lumen 116 may extend from a position adjacent the proximal end 111 of the catheter 110 to a position adjacent the distal end 112 of the catheter 110. In other embodiments, the catheter lumen 116 may only extend along a portion of the length of the catheter 110 and may be accessed through openings or ports in the sidewall of the catheter 110 in addition to, or in place of, openings in the proximal 111 or distal 112 ends of the catheter 110.

The illustrated embodiment further comprises a first port 117 and a second port 118 coupled adjacent the proximal end 111 of the catheter 110. Other embodiments within the scope of this disclosure may comprise only one or neither of this ports. The first 117 and second 118 ports may be in communication with the catheter lumen 116 and may be used to provide fluidic or mechanical access to the catheter lumen 116 and/or the distal end 112 of the catheter 110. In some embodiments, one or both of the first 117 and second 118 ports may be in communication with a secondary and/or tertiary lumen disposed within the catheter 110 which may not be in communication with the catheter lumen 116. Moreover, catheters that have additional ports and/or lumens are within the scope of this disclosure. In the illustrated embodiment, the catheter 110 further comprises a catheter lumen port 119. The catheter lumen port 119 may be disposed adjacent the proximal end 111 of the catheter 110 (as shown in the illustrated embodiment) or it may be disposed in a sidewall of the catheter 110 at another position along the catheter body 115.

The assembly 100 may further comprise a dilator 120 extending from a proximal end 121 to a distal end 122 with a dilator body 125 disposed there between. A dilator lumen 126 may be disposed within the dilator 120, extending from the proximal end 121 to the distal end 122 therefore, or may extend along a portion of the dilator 120. The dilator 120 may be configured with openings in the proximal 121 and distal 122 ends to provide communication with the dilator lumen 126. Additionally or alternatively, sidewall openings in the dilator body 125 may be in communication with the dilator lumen 126.

The dilator 120 may be disposed within, and axially displaceable along, the catheter lumen 116. In some embodiments the dilator 120 may be longer than the catheter 110 such that both the proximal 121 and distal 122 ends of the dilator 120 may extend beyond the proximal 111 and distal 112 ends of the catheter 110 when the dilator 120 is disposed within the catheter lumen 116. Thus, displacement of the proximal end 121 of the dilator 120 (to which a practitioner may have more direct access) may be configured to axially displace the dilator 120 within the catheter lumen 116. The distal end 122 of the dilator 120 may be configured to provide a transition between smaller-diameter items (such as the needle 130 or guidewire 140) and the larger diameter of the distal end 112 of the catheter 120.

The assembly 100 may also comprise a needle 130, which may be disposed within the dilator lumen 126. Because the dilator 120 may be disposed within the catheter lumen 116 while the needle 130 is disposed within the dilator lumen 126, the needle 130 may also be described as disposed within the catheter lumen 116. This convention applies throughout the present disclosure; any disclosure indicating that a first component may be disposed within the lumen of a second component does not necessarily imply that no intermediate component is present. The assembly 100 may comprise various components which may be coaxially aligned such that a single component may be simultaneously disposed within the lumens of more than one of the surrounding components.

The needle 130 may be axially displaceable within the dilator lumen 126 and the catheter lumen 116. In the illustrated embodiment, a needle wire 135 is coupled to a proximal end of the needle 130. In the position shown in the illustrated embodiment, this coupling is at a position within the dilator lumen 126, as shown in FIG. 2. Agin referring to FIGS. 1-3, the needle wire 135 may extend proximally from the needle 130 such that the needle wire extends from proximal openings in the dilator lumen 126 and/or the catheter lumen 116. Displacement of the proximal end 131 of the needle wire (to which a practitioner may have more direct access) 135 may be configured to displace the needle wire 135 and needle 130 within the dilator lumen 126 and/or the catheter lumen 116.

In the illustrated embodiment, the assembly further comprises a guidewire 140 extending from a proximal end 141 to a distal end 142 with a guidewire body 145 disposed there between. The guidewire 140 may be disposed within the needle lumen 136 as well as within the dilator lumen 126 and catheter lumen 116. As shown FIG. 2, the guidewire 140 may extend from the needle lumen 136 at a position within the dilator lumen 126 and/or the catheter lumen 116. Again referring to FIGS. 1-3, the guidewire 140 may be axially displaceable within these lumens and may have sufficient length that the proximal 141 and distal 142 ends of the guidewire 140 may simultaneously extend from the needle lumen at the distal end 132 of the needle 130 as well as from the dilator lumen 126 and/or catheter lumen 116 at the proximal ends of the dilator 120 and/or catheter 110. Displacement of the proximal end 141 of the guidewire 140 may be configured to axially displace the guidewire 140 within the lumens of the other components of the assembly 100. The guidewire 140 may further comprise a marking tag 149 disposed near or adjacent the proximal end 141 of the guidewire 140. The marking tag 149 may be configured to distinguish the guidewire 140 from the needle wire 135. The needle wire 135 may likewise have a marking tag 139 adjacent or near the proximal end 131 of the needle wire 135. These marking tags 139, 149 may be removable to accommodate removal of components over the wires (for example removal of the needle 130 proximally over the guidewire 140). Further, other embodiments include other indicia (i.e., the color or texture of the wires) in addition to, or in place of, the marking tags 139, 149.

The components of the assembly 100 may generally be coaxially aligned, as shown in FIGS. 2 and 3, and each component may be axially displaceable with respect to the other components. Again referring to FIGS. 1-3, manipulation of the various components at or near the proximal ends thereof may be configured to displace those components along or within the assembly 100. For example, during some procedures the distal ends of the various components may be disposed within the body of a patient. Further, the body portions of some of the components may be disposed within lumens of other components. Thus, manipulation of one or any component at the proximal ends thereof may allow a practitioner to manipulate the components within or along the assembly 100 and/or within the patient's body.

In one exemplary procedure, a practitioner may utilize the assembly 100 as part of a vascular access procedure, such as vascular cannulation. The practitioner may initially guide the distal end 132 of the needle 130 through the skin 50 and into a vascular lumen 70 of a patient. Ultrasound or other imaging techniques may be utilized to guide and confirm placement of the needle 130 within the vascular lumen 70. During insertion of the needle 130 into the vascular lumen 70, the guidewire 140 may be disposed within the needle lumen 136, but may not extend from the distal end 132 of the needle 130. In some instances, the presence of the guidewire 140 within the needle lumen 136 may make the needle 130 more echogenic, allowing it to be more easily viewed through ultrasound. Further, movement of the guidewire 140 within the needle lumen 136 may be utilized to aid in locating the needle 130 through ultrasound.

Once the needle 130 is placed within the vascular lumen 70, the guidewire 140 may be advanced such that the distal end 142 of the guidewire 140 extends from the needle 130, essentially in the position shown in the illustrated embodiment. With the guidewire 140 disposed within the vascular lumen 70, the needle 130 may then be removed by proximally displacing the needle 130 over the guidewire 140. The needle 130 may be fully removed from the assembly 100 by displacing the needle 130 proximally such that the needle 130 is pulled over the proximal end 141 of the guidewire 140.

The dilator 120 and catheter 110 may then be advanced over the guidewire 140 until the dilator 120 and catheter 110 are disposed within the vascular lumen 70. In some procedures the dilator 120 and catheter 110 may be advanced simultaneously, while in others the dilator 120 may be advanced prior to the catheter 110. The dilator 120 may be configured to provide a transition between the catheter 110 and the smaller opening in the skin 50, as well as to provide a transition between the catheter 110 and the smaller diameter guidewire 140 extending through the skin 50.

In some embodiments, the catheter 110 may be advanced without use of a dilator 120. For example, the catheter 110, needle 130, and guidewire 140 may be sized such that a transition component is needed. Further, the catheter 110 may be configured with an angled portion adjacent the distal end 112 of the catheter 110, which may obviate the need for a dilator 120. Assemblies such as assembly 100 having no dilator 120 are within the scope of this disclosure.

Whether the dilator 120 and catheter 110 are advanced together, separately, or with no dilator 120, in some embodiments a practitioner may make a small incision or nick in the skin 50 adjacent the entry site to facilitate passage of the dilator 120, catheter 110, or both. Additionally, a practitioner may advance the dilator 120, catheter 110, or both prior to withdrawing the needle 130 over the guidewire 140. Thus, in some embodiments, the dilator 120 and/or catheter 110 may be advanced over the guidewire 140 and/or the needle 130.

Once the catheter 110 is advanced into the vascular lumen 70, the dilator 120 and guidewire 140 (and needle 130 if not previously withdrawn) may be withdrawn proximally from the assembly 100. The catheter 110 may thus be properly placed for use as part of any additional therapy or procedure.

The method discussed above may obviate the use of a syringe during cannulation and may not depend on the presence of a “flash” of blood. Additionally, as the method discussed above may not therefore require a practitioner to remove the syringe from the needle, the guidewire may be immediately advanced into the vascular lumen once the needle is placed, without intermediate steps. This may lessen the risk of needle dislodgement before the guidewire is advanced. Further, the assembly described herein may be generally easier to operate and less cumbersome than kits having additional parts and/or requiring additional steps.

The examples and embodiments disclosed herein are to be construed as merely illustrative and exemplary, and not a limitation of the scope of the present disclosure in any way. It will be apparent to those having skill in the art with the aid of the present disclosure that changes may be made to the details of the above-described embodiments without departing from the underlying principles of the disclosure herein. It is intended that the scope of the invention be defined by the claims appended hereto and their equivalents.

1. A catheter assembly comprising:
an elongate catheter extending from a proximal end to a distal end with a catheter lumen disposed therein;
a needle disposed within the catheter lumen such that the needle is axially displaceable, by a practitioner, within the catheter lumen, the needle comprising a needle lumen; and
a guidewire disposed within the needle lumen such that the guidewire is axially displaceable, by a practitioner, within the needle lumen.

2. The catheter assembly of claim 1, further comprising a needle wire coupled to a proximal end of the needle, the needle wire extending through the catheter lumen such that a distal end of the needle wire extends from a distal end of the catheter lumen.

3. The catheter assembly of claim 1, wherein a distal end of the guidewire extends from a distal end of the catheter lumen.

4. The catheter assembly of claim 1, further comprising a dilator disposed within the catheter lumen such that the dilator is axially displaceable, by a practitioner, within the catheter lumen, the dilator comprising a dilator lumen, and wherein the needle and the guidewire are disposed within the dilator lumen.

5. The catheter assembly of claim 4, wherein a distal end of the dilator extends from a distal end of the catheter lumen.

6. The catheter assembly of claim 4, further comprising a needle wire coupled to a proximal end of the needle, the needle wire extending through the dilator lumen such that a distal end of the needle wire extends from a distal end of the dilator lumen.

7. The catheter assembly of claim 4, wherein a distal end of the guidewire extends from a distal end of the dilator lumen.

8. A method of vascular access, the method comprising:
extending a needle through a patient's skin into a vascular lumen wherein a proximal portion of the needle is disposed within a catheter lumen of a catheter;
advancing a guidewire through a needle lumen such that a distal end of the guidewire is disposed within the vascular lumen and a proximal portion of the guidewire is disposed within the catheter lumen;
proximally displacing the needle to withdraw the needle from the vascular lumen and from the catheter lumen; and
advancing the catheter distally along the guidewire until a distal end of the catheter is disposed within the vascular lumen.

9. The method of claim 8, further comprising proximally displacing the guidewire to withdraw the guidewire from the vascular lumen and from the catheter lumen.

10. The method of claim 8, wherein the step of extending the needle into the vascular lumen comprises guiding the needle by ultrasound.

11. The method of claim 8, further comprising axially displacing the guidewire within the needle lumen to increase the echogenicity of the needle to ultrasound.

12. The method of claim 8, further comprising nicking the patient's skin at the needle insertion point to facilitate passage of the catheter through the skin.

13. The method of claim 8, further comprising advancing a dilator along the guidewire until a distal end of the dilator is disposed within the vascular lumen, the dilator disposed within the catheter lumen and configured to facilitate passage of the catheter into the vascular lumen.

14. The method of claim 13, further comprising proximally displacing the dilator to withdraw the dilator from the vascular lumen and from the catheter lumen.

15. The method of claim 8, wherein the step of proximally displacing the needle comprises proximally displacing a needle wire extending from a proximal end of the catheter lumen.

16. The method of claim 9, wherein the step of proximally displacing the guidewire comprises proximally displacing a proximal end of the guidewire extending from a proximal end of the catheter lumen.

17. The method of claim 14, wherein the step of proximally displacing the dilator comprises proximally displacing a proximal end of the dilator extending from a proximal end of the catheter lumen.
